(19) United States
(12) Patent Application Publication
(10) Pub. No.: US 2026/0248444 A1
(43) Pub. Date: Aug. 27, 2026

(54) MEASURING DEVICE FOR BIOIMPEDANCE SPECTROSCOPY OF THE BREAST

(71) Applicant: XERAMED GMBH, Mauren (LI)

(72) Inventors: Jörg GRABOW, Königsee (DE); Horst SCHWAB, Radebeul (DE)

(73) Assignee: XERAMED GMBH, Mauren (LI)

(21) Appl. No.: 19/136,588

(22) PCT Filed: Dec. 8, 2023
(86) PCT No.: PCT/EP2023/084885

§ 371 Date: Jun. 6, 2025

(30) Foreign Application Priority Data
Dec. 9, 2022 (EP) 22212648.4

(51) Int. Cl. A61B 5/00; A61B 5/053

(52) U.S. Cl. CPC A61B 5/4312; A61B 5/053; A61B 5/6823

(57) ABSTRACT

A bioimpedance measuring device for measuring breast tissue is provided, comprising a control unit and at least one measuring applicator which comprises at least four contact electrodes which are connected or connectable to the control unit via electrical lines, wherein the control unit is designed to generate an alternating current electrical signal and to output it to two of the at least four contact electrodes so that these act as current electrodes, and to receive an alternating voltage signal between two of the at least four contact electrodes, so that these act as voltage electrodes, wherein at least two of the contact electrodes which act as voltage electrodes do not simultaneously act as current electrodes.

The invention relates to a measuring device and a method for measuring a bioimpedance profile of human breast tissue.

In Germany and other countries, breast cancer is considered to be one of the main causes of disability and one of the most common causes of death among women. Around 72,000 women in Germany develop breast cancer every year, and around 17,000 women die of this disease every year.

The treatment prospects for breast cancer depend heavily on the stage at which the disease is detected. Early detection is therefore one of the most effective methods of containing the disease.

Common methods for the early detection of breast cancer include palpation, ultrasound, and mammography. While palpation and ultrasound examinations are often unable to reliably identify small tumors, mammography is extremely uncomfortable for the patient and, due to the associated radiation exposure and tissue compression, itself represents a risk factor for adverse development of pathologically altered breast tissue.

For some time now, methods have been known that use electrical impulses to measure the tissue composition of the human body. To do this, small alternating currents are passed through the body tissue, and the impedance of the tissue is determined. The currents used are so small that no irritation of the tissue occurs. This method, known as bioimpedance measurement, is used primarily to determine the body fat percentage in nutritional counselling.

The object of the invention is therefore to provide an alternative method for the early detection of tissue changes in human breast tissue, so that further differential diagnostic tests can be carried out in good time and, if necessary, an early therapy can be initiated.

A further object of the invention is to provide a suitable measuring device for a corresponding alternative method.

According to one aspect of the present invention, the problem is solved by a bioimpedance measuring device for measuring breast tissue, with a control unit and at least one measuring applicator, which comprises at least four contact electrodes, which are connected or can be connected to the control unit via electrical lines, wherein the control unit is set up to generate an electrical alternating current signal and to two of the at least four contact electrodes so that they function as current electrodes, and to receive an alternating electrical voltage signal between two of the at least four contact electrodes so that they function as voltage electrodes, wherein at least two of the contact electrodes that function as voltage electrodes do not simultaneously function as current electrodes.

The invention is based on the finding that a localized measurement of the breast tissue is possible by means of a bioimpedance measurement of the breast tissue via suitably arranged contact electrodes.

In an advantageous configuration of the bioimpedance measuring device, the contact electrodes of the at least one measuring applicator are arranged at the corners of a regular polygon. A corresponding arrangement simplifies a reproducible execution of the bioimpedance measurement, so that the results of measurements carried out at intervals can be easily compared. Preferably, the measuring applicator comprises four contact electrodes arranged at the corners of a square.

In one possible embodiment, the measuring applicator comprises a carrier region with arm-like extensions, at the ends of which the contact electrodes are arranged. This design allows the measuring applicator to be applied to the breast tissue to be examined with a particularly small contact area.

The carrier region can include an opening that is arranged at a midpoint of the regular polygon. The measuring applicator can be placed on the breast tissue in such a way that the nipple remains visible through the opening. In this way, reproducible positioning of the measuring applicator is easy to achieve for repeated measurements.

The carrier region of the measuring applicator can preferably be made of a flexible material so that the measuring applicator can be placed flexibly against the breast tissue. A suitable material with high biocompatibility is, for example, silicone.

In a preferred embodiment, the contact electrodes are detachably connected to the measuring applicator. This means, for example, that single-use contact electrodes can be used with a reusable measuring applicator.

In one possible configuration, the control unit of the bioimpedance measuring device is set up to generate the alternating current signal at a variable frequency. By measuring the bioimpedance at different frequencies, it is possible to obtain a more precise characterization of the breast tissue being measured.

The control unit is also preferably set up to alternately control different contact electrodes as voltage electrodes. The alternating control of the contact electrodes allows a spatially resolved bioimpedance profile of the tissue to be examined.

In an advantageous embodiment of the invention, a bioimpedance measuring device can also include a computing unit that is set up to calculate a spatial impedance distribution of the examined breast tissue from the alternating voltage signals recorded at different frequencies and/or with different electrode configurations.

The control unit preferably includes a memory device and is set up to store the calculated impedance distribution and/or the alternating voltage signals recorded at different frequencies and/or with different electrode configurations.

In one possible embodiment, the control unit of the bioimpedance measuring device is arranged in a mobile housing. It can thus be easily positioned in relation to an examination couch on which the person to be examined is lying. A medical equipment trolley, for example, can be used as the mobile housing.

The mobile housing can include an extension arm along which the electrical lines are or can be routed. This can prevent the electrical lines from lying on the skin surface of the person to be examined.

In a particularly advantageous embodiment, the bioimpedance measuring device can include two measuring applicators with which both breasts of a person to be examined can be measured simultaneously.

The task is further solved by a method for recording a bioimpedance profile of human breast tissue, comprising the steps of: Applying a measuring applicator with at least four contact electrodes to the breast tissue to be examined, feeding an alternating current signal via two of the at least four contact electrodes, which act as current electrodes, and recording an alternating voltage signal across two of the at least four contact electrodes, which act as voltage electrodes, wherein a frequency of the alternating current signal is varied over a predetermined frequency range, and wherein a function of each of the at least four contact electrodes is alternated between current electrode and voltage electrodes. The method according to the invention makes it possible to create a spatially resolved bioimpedance profile of the breast tissue.

In a preferred embodiment of the method according to the invention, the measuring applicator comprises four contact electrodes arranged at the corners of a square, wherein in a first measuring interval a first and a second contact electrode function as current electrodes and a third and a fourth contact electrode function as voltage electrodes, in a second measuring interval the first and the third contact electrode function as current electrodes and the second and the fourth contact electrodes act as voltage electrodes, in a third measuring interval the first and fourth contact electrodes act as current electrodes and the second and third contact electrodes act as voltage electrodes, in a fourth measuring interval the second and third contact electrodes act as current electrodes and the first and fourth contact electrodes act as voltage electrodes, in a fifth measuring interval the second and fourth contact electrodes function as current electrodes and the first and third contact electrodes function as voltage electrodes, and in a sixth measuring interval the third and fourth contact electrodes function as current electrodes and the first and second contact electrodes function as voltage electrodes.

In a further preferred embodiment of the method according to the invention, the frequency of the alternating current signal is varied in each measuring interval in several frequency steps in a frequency range between 1 kHz and 1 MHz.

In each of the measuring intervals and for each frequency step, an amplitude and a phase of the voltage signal can be measured. In this way, a spatially and frequency-resolved bioimpedance profile of the tissue to be examined can be created. For this purpose, the recorded measurement values are stored.

A bioimpedance profile stored in this way can be compared with a previously recorded bioimpedance profile of the same tissue. The previously recorded bioimpedance profile may have been recorded during a previous regular check-up.

If the comparison of the bioimpedance profile with the previously recorded bioimpedance profile shows a change that exceeds a predetermined level, a change signal can be generated.

This change signal can indicate the need to initiate further differential diagnostic steps in order to rule out or confirm the presence of a pathological tissue change.

The invention will be explained in more detail below with the aid of a few exemplary illustrations. The illustrations serve only to provide a better understanding of the invention without restricting it.

The following shows:

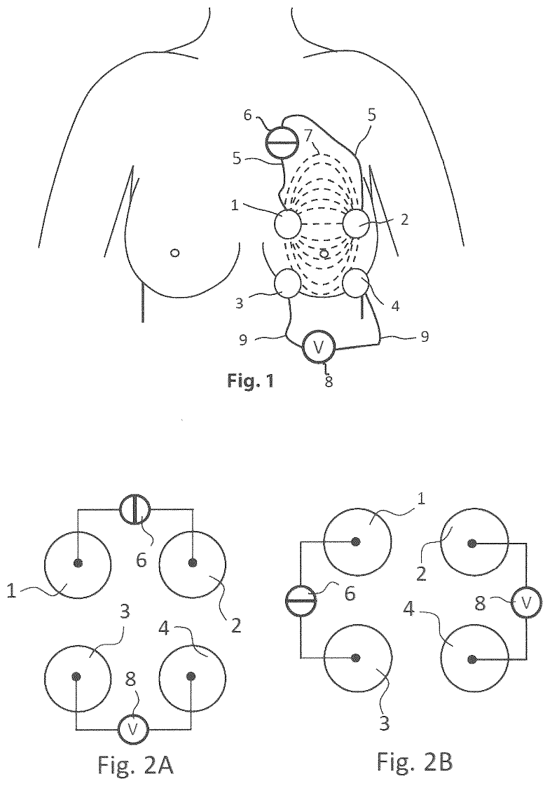

FIG. 1: a schematic representation of the bioimpedance measurement of breast tissue,

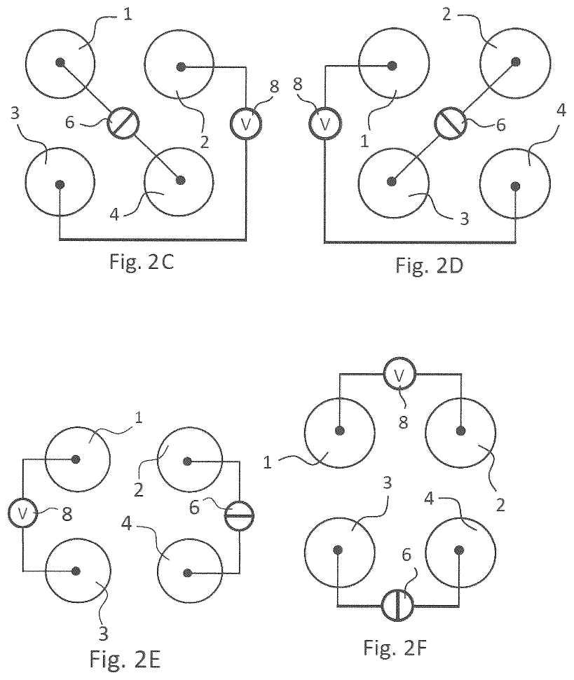

FIG. 2A-2F: connection configurations of the contact electrodes,

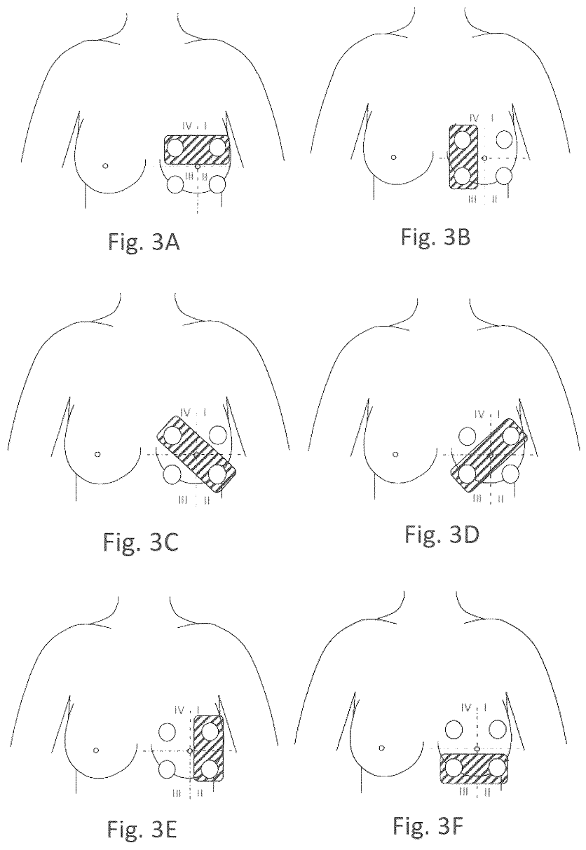

FIG. 3A-3F: examination areas of the connection configurations from FIGS. 2A-2F,

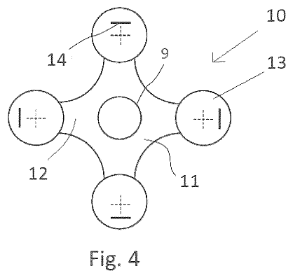

FIG. 4: a measuring applicator,

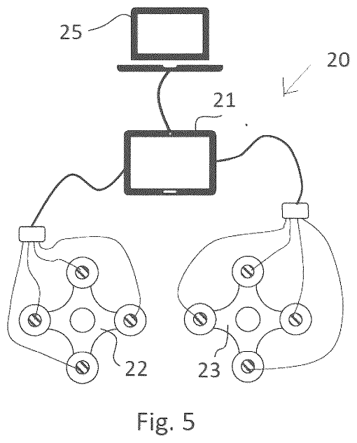

FIG. 5: a bioimpedance measuring system,

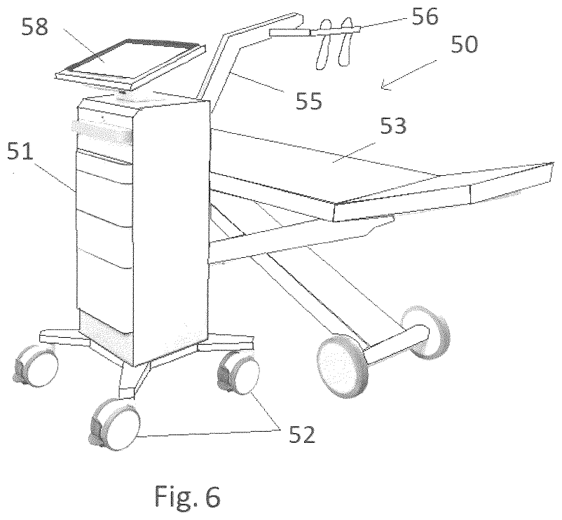

FIG. 6: an examination system.

FIG. 1 shows a female torso with indicated breast tissue, which is to be subjected to a bioimpedance measurement. For this purpose, four contact electrodes 1, 2, 3, 4 are attached to the left breast. The contact electrodes 1, 2 are connected to an alternating current source 6 via electric lines 5; they therefore act as current electrodes. The alternating current source 6 introduces a current signal in the form of a weak high-frequency alternating current into the breast tissue to be examined. This creates an electromagnetic field in the breast tissue, which is indicated by the field lines 7. The electromagnetic field creates a potential difference between the contact electrodes 3, 4, which is measured by the voltmeter 8. The voltmeter 8 is connected to the contact electrodes 3, 4 by means of electric leads 9, which therefore act as voltage electrodes.

The path of the electromagnetic field lines 7, which is shown in a greatly simplified way in FIG. 1, depends on the frequency of the impressed alternating current and on the composition of the breast tissue in the area between the current electrodes 1, 2 and the voltage electrodes 3, 4. By changing the frequency of the impressed alternating current, the composition of the breast tissue in the measuring area can be determined.

For the bioimpedance measurement described here, it is not necessary to actually determine the physiological tissue composition. Rather, it is sufficient to record the course of the amplitude and phase of the voltage signal as a function of the frequency of the current signal. This is because it is sufficient for the early detection of tissue changes to recognize a change in the physiological tissue composition, which is reflected in a change in the course of the amplitude and phase of the voltage signal.

FIGS. 2A to 2F show how the contact electrodes 1, 2, 3, 4 are connected in succession to the alternating current source 6 and to the voltage measuring device 8 when the impedance profile is recorded.

FIG. 2A shows the configuration already indicated in FIG. 1, in which contact electrodes 1 and 2 are connected to the alternating current source 6 as current electrodes, while contact electrodes 3 and 4 are connected to the voltmeter 8 as voltage electrodes.

FIG. 2B shows a configuration in which contact electrodes 1 and 3 are connected to the alternating current source 6 as current electrodes, while contact electrodes 2 and 4 are connected to the voltmeter 8 as voltage electrodes.

FIG. 2C shows a configuration in which contact electrodes 1 and 4 are connected to the alternating current source 6 as current electrodes, while contact electrodes 2 and 3 are connected to the voltmeter 8 as voltage electrodes.

FIG. 2D shows a configuration in which contact electrodes 2 and 3 are connected to the alternating current source 6 as current electrodes, while contact electrodes 1 and 4 are connected to the voltage measuring device 8 as voltage electrodes.

FIG. 2E shows a configuration in which contact electrodes 2 and 4 are connected to the alternating current source 6 as current electrodes, while contact electrodes 1 and 3 are connected to the voltmeter 8 as voltage electrodes.

FIG. 2F shows a configuration in which contact electrodes 3 and 4 are connected to the alternating current source 6 as the current electrode, while contact electrodes 1 and 2 are connected to the voltmeter 8 as the voltage electrodes.

FIGS. 3A to 3F show the areas of breast tissue through which the alternating current signal flows with the contact electrode configurations shown in FIGS. 2A to 2F. The four sectors of the breast are labelled I-IV.

FIG. 4 shows a measuring applicator 10 of a bioimpedance measuring device according to the invention. The measuring applicator comprises a carrier region 11, which comprises four arm-like extensions 12, at the ends of which holders 13 for contact electrodes are arranged. Contact electrodes, which are not shown in FIG. 4, can be introduced into the holders 13 via slots 14 in the holders 13, so that they come into contact with the breast tissue to be examined when the measuring applicator 10 is placed on it. The carrier region 11 with the extensions 12 is made of a flexible and biocompatible material such as silicone. The extensions 12 therefore gently make contact with the breast tissue to be examined when the measuring applicator 10 is placed on it, without it being necessary to exert pressure on the measuring applicator 10.

In the center of the measuring applicator 10, an opening 15 is provided through which the nipple remains visible when the measuring applicator 10 is placed on the breast tissue. This makes it easier to place the measuring applicator 10 in the same position on the breast tissue to be examined for successive measurements.

FIG. 5 shows a bioimpedance measurement system 20. The bioimpedance measurement system 20 includes a control unit 21, which contains an alternating current source and a voltmeter, which for the sake of clarity are not shown separately. Two measuring applicators 22, 23 are connected to the control unit 21, which are designed like the measuring applicator 10 shown in FIG. 4. With the two measuring applicators 22, 23, both breasts of a person to be examined can be measured simultaneously.

The bioimpedance measurement system 20 is additionally connected to a computer 25, by means of which the phases and amplitudes of the voltage signal recorded at different frequencies of the alternating current signal and at different electrode configurations are logged and evaluated to form a spatial impedance distribution of the breast tissue under examination. A spatial impedance distribution in this context does not necessarily mean an exact spatially resolved impedance distribution in which a separate impedance measurement is available for each spatial element of the examined breast tissue in the sense of a tomographic slice. In the context of the present invention, a spatial impedance distribution is also understood to mean a multidimensional characteristic curve field in which the amplitude and phase of the voltage signal are plotted over the different electrode configurations and different measuring frequencies.

The control unit 21 can, for example, vary the frequency of the alternating current signal in several steps between 1 kHz and 1 MHz for each electrode configuration. The frequency can, for example, be increased in steps of 1 kHz, so that after 1000 steps the frequency of 1 MHz is reached. A short measurement time of, for example, 0.1 seconds is sufficient for each frequency step to determine the measured values for the phase and the amplitude of the alternating voltage. Less than two minutes are therefore required for the 1000 frequency steps, so that the complete measurement of the breast tissue with all six electrode configurations can be completed in about ten minutes.

The measurement results can be analyzed by the computer immediately after the measurement. To analyze the measurement results, reference values can be used that represent normal progressions of the measured values in healthy tissue. However, since such reference values can only be rough guidelines, it makes sense to compare the measurement results of one examination with the measurement results of a previous examination of the same breast tissue. If, for example, a bioimpedance measurement according to the invention is carried out regularly at yearly intervals, suspicious changes in the tissue composition can be detected relatively early, even before there is a palpable or ultrasound-visible tissue change.

An exemplary examination system for carrying out the bioimpedance measurement according to this invention is shown in FIG. 6. The examination system 50 comprises an equipment trolley 51, in which the bioimpedance measurement system 20 shown in FIG. 5 is integrated. The equipment trolley 51 is provided with casters 52 for positioning it to an examination couch 53 on which the person to be examined can be horizontally positioned. Furthermore, an extension arm 55 is provided on the equipment trolley 51, along which electrical lines from the control unit 21, which is not shown in FIG. 6, run. At the end of the extension arm 55 that is close to the patient, a connection area 56 is provided in which the electrical lines are freely accessible. Eight electrical lines are provided for connecting two measuring applicators with four contact electrodes each. For the sake of clarity, only two lines are shown in FIG. 6.

The extension arm 55 can be positioned above the person to be examined in such a way that the measuring applicators not shown in FIG. 6 can be placed on both sides of the chest tissue without the electrical cables having to be placed on the skin of the person to be examined in a disruptive manner. The free ends of the electrical lines can be connected to single-use contact electrodes, which are then inserted into the respective holders of the measuring applicators.

A control panel 58 on the equipment trolley 61 is used to operate the bioimpedance measurement system by a user. The control panel 58 can be connected to the computer 25 or be part of the computer.

The following sections describe different embodiments of the invention.
1. Bioimpedance measuring device for measuring breast tissue, with a control unit and at least one measuring applicator having at least four contact electrodes which are connected or connectable to the control unit via electrical lines, wherein the control unit is designed to generate an alternating current electrical signal and output it to two of the at least four contact electrodes so that they function as current electrodes, and to receive an alternating voltage electrical signal between two of the at least four contact electrodes so that they function as voltage electrodes, wherein at least two of the contact electrodes functioning as voltage electrodes do not simultaneously function as current electrodes.
2. Bioimpedance measuring device according to section 1, wherein the contact electrodes of the at least one measuring applicator are arranged at the corners of a regular polygon.
3. Bioimpedance measuring device according to section 1 or 2, wherein the measuring applicator has four contact electrodes.
4. Bioimpedance measuring device according to section 2 or 3, wherein the measuring applicator comprises a carrier region with arm-like extensions at the ends of which the contact electrodes are arranged.
5. Bioimpedance measuring device according to one of sections 2 to 4, wherein the carrier region comprises an opening arranged at a center point of the regular polygon and enabling reproducible positioning of the measuring applicator on the nipple of a breast to be measured.
6. Bioimpedance measuring device according to section 4 or 5, wherein the carrier region is made of a flexible material.
7. Bioimpedance measuring device according to one of the preceding sections, wherein the contact electrodes are detachably connected to the measuring applicator.
8. Bioimpedance measuring device according to one of the preceding sections, wherein the control device is designed to generate the alternating current signal with variable frequency.
9. Bioimpedance measuring device according to one of the preceding sections, wherein the control device is designed to alternately control different contact electrodes as current electrodes.
10. Bioimpedance measuring device according to one of the preceding sections, wherein the control device is designed to alternately control different contact electrodes as voltage electrodes.
11. Bioimpedance measuring device according to one of the preceding sections, wherein the control device comprises a computing unit which is designed to calculate a spatial impedance distribution of the examined breast tissue from the alternating voltage signals recorded at different frequencies and/or with different electrode configurations.
12. Bioimpedance measuring device according to one of the preceding sections, wherein the control device comprises a memory device and is designed to store the calculated impedance distribution and/or the alternating voltage signals recorded at different frequencies and/or with different electrode configurations.
13. Bioimpedance measuring device according to one of the preceding sections, wherein the control device is arranged in a mobile housing.
14. Bioimpedance measuring device according to section 13, wherein the mobile housing comprises an extension arm along which the electrical lines are guided or can be guided.
15. Bioimpedance measuring device according to one of the preceding sections, wherein the bioimpedance measuring device comprises two measuring applicators.
16. Measuring applicator of a bioimpedance measuring device according to one of sections 1 to 7.
17. Method for recording a bioimpedance profile of human breast tissue, comprising the steps of applying a measuring applicator with at least four contact electrodes to the breast tissue to be examined, feeding in an alternating current signal via two of the at least four contact electrodes, which act as current electrodes, and recording an alternating voltage signal via two of the at least four contact electrodes, which function as voltage electrodes, wherein a frequency of the alternating current signal is varied over a predetermined frequency range, and wherein a function of each of the at least four contact electrodes is changed between current electrode and voltage electrode.
18. Method according to section 17, wherein the measuring applicator comprises four contact electrodes arranged at the corners of a square, and wherein, in a first measurement interval, a first and a second contact electrode function as current electrodes, and a third and a fourth contact electrode function as voltage electrodes, in a second measurement interval, the first and the third contact electrode function as current electrodes, and the second and the fourth contact electrode function as voltage electrodes, in a third measuring interval, the first and fourth contact electrodes function as current electrodes, and the second and third contact electrodes function as voltage electrodes, in a fourth measuring interval, the second and third contact electrodes function as current electrodes, and the first and fourth contact electrodes function as voltage electrodes, in a fifth measurement interval, the second and fourth contact electrodes function as current electrodes, and the first and third contact electrodes function as voltage electrodes, and in a sixth measurement interval, the third and fourth contact electrodes function as current electrodes, and the first and second contact electrodes function as voltage electrodes.
19. Method according to section 18, wherein the frequency of the alternating current signal is varied in each measurement interval in several frequency steps in a frequency range between 1 kHz and 1 MHz.
20. Method according to section 19, wherein an amplitude and a phase of the voltage signal are measured in each measurement interval and at each frequency step.
21. Method according to any of sections 17 to 20, wherein the recorded measured values are stored as a bioimpedance profile.
22. Method according to section 21, wherein the stored bioimpedance profile is compared with a previously recorded bioimpedance profile of the same tissue.
23. Method according to section 22, wherein changes in the bioimpedance profile which exceed a predetermined level generate a change signal.

1. Bioimpedance measuring device for measuring breast tissue, comprising a control unit and at least one measuring applicator having a first, a second, a third and a fourth contact electrode, which are arranged at the four corners of a square and which are or can be connected to the control unit via electrical lines, wherein the control unit is configured to
generating an alternating current electrical signal and outputting it to two of the four contact electrodes so that they function as current electrodes, and
receiving an alternating voltage electrical signal between two of the four contact electrodes so that they function as voltage electrodes,
wherein at least two of the contact electrodes functioning as voltage electrodes do not simultaneously function as current electrodes,
the control unit being further configured to apply an alternating electrical current signal in succession
simultaneously to the first and second contact electrodes,
simultaneously to the first and third contact electrodes,
simultaneously to the first and fourth contact electrodes,
simultaneously to the second and third contact electrodes,
simultaneously to the second and fourth contact electrodes,
simultaneously to the third and fourth contact electrodes,
the control unit comprises a computing unit that is configured to calculate a spatial impedance distribution of the breast tissue under examination from the alternating voltage signals recorded with different electrode configurations.

2. Bioimpedance measuring device according to claim 1, characterized in that the measuring applicator comprises a carrier region with arm-like extensions, at the ends of which the contact electrodes are arranged.

3. Bioimpedance measuring device according to claim 1, characterized in that the carrier region comprises an opening which is arranged at a midpoint of the regular polygon, and to enable reproducible positioning of the measuring applicator on the nipple of the breast to be measured.

4. Bioimpedance measuring device according to claim 2, characterized in that the carrier region consists of a flexible material.

5. Bioimpedance measuring device according to claim 1, characterized in that the contact electrodes are detachably connected to the measuring applicator.

6. Bioimpedance measuring device according to claim 1, characterized in that the control unit is configured to generate the alternating current signal with variable frequency.

7. Bioimpedance measuring device according to claim 1, characterized in that the control unit is configured to alternately control different contact electrodes as current electrodes.

8. Bioimpedance measuring device according to claim 1, characterized in that the control unit is configured to alternately control different contact electrodes as voltage electrodes.

9. Bioimpedance measuring device according to claim 1, characterized in that the computing unit is configured to calculate a spatial impedance distribution of the examined breast tissue from the alternating voltage signals recorded at different frequencies.

10. Bioimpedance measuring device according to claim 1, characterized in that the control unit comprises a memory device and is configured to store the calculated impedance distribution and/or the alternating voltage signals recorded at different frequencies and/or with different electrode configurations.

11. Bioimpedance measuring device according to claim 1, characterized in that the control unit is arranged in a mobile housing.

12. Bioimpedance measuring device according to claim 11, characterized in that the mobile housing comprises an extension arm along which the electric lines are guided or can be guided.

13. Bioimpedance measuring device according to claim 1, characterized in that the bioimpedance measuring device comprises two measuring applicators.

14. Measuring applicator of a bioimpedance measuring device according to claim 1.

15. Method for recording a bioimpedance profile of human breast tissue, comprising the steps of:
applying a measuring applicator having four contact electrodes to the breast tissue to be examined,
feeding an alternating current signal via two of the four contact electrodes, which act as current electrodes, and
recording an alternating voltage signal via two of the four contact electrodes, which act as voltage electrodes,
wherein a frequency of the alternating current signal is carried over a predetermined frequency range, and
wherein a function of each of the four contact electrodes is alternated between current electrode and voltage electrode, wherein the measurement applicator comprises four contact electrodes arranged at the corners of a square, and wherein
in a first measuring interval, a first and a second contact electrode function as current electrode and a third and a fourth contact electrode function as voltage electrode,
in a second measuring interval, the first and the third contact electrode function as current electrode and the second and the fourth contact electrode function as voltage electrode,
in a third measuring interval, the first and fourth contact electrodes act as current electrodes and the second and third contact electrodes act as voltage electrodes,
in a fourth measuring interval, the second and third contact electrodes act as current electrodes and the first and fourth contact electrodes act as voltage electrodes,
in a fifth measuring interval, the second and fourth contact electrodes function as current electrodes, and the first and third contact electrodes function as voltage electrodes, and
in a sixth measuring interval, the third and fourth contact electrodes function as current electrodes, and the first and second contact electrodes function as voltage electrodes, and wherein
a spatial impedance distribution of the examined breast tissue is calculated by means of a computing unit from the alternating voltage signals recorded with different electrode configurations.

16. Method according to claim 15, characterized in that the frequency of the alternating current signal is varied in each measuring interval in a plurality of frequency steps in a frequency range between 1 kHz and 1 MHz.

17. Method according to claim 16, characterized in that an amplitude and a phase of the voltage signal are measured in each measuring interval and a each frequency step.

18. Method according to claim 15, characterized in that the measured values recorded are stored as a bioimpedance profile.

19. Method according to claim 18, characterized in that the stored bioimpedance profile is compared with a previously recorded bioimpedance profile of the same tissue.

20. Method according to claim 19, characterized in that, when changes in the bioimpedance profile which exceed predetermined level, a change signal is generated.
